(19) United States
(12) Patent Application Publication
(10) Pub. No.: US 2023/0301741 A1
(43) Pub. Date: Sep. 28, 2023

(54) MOBILE PERSONAL PROTECTIVE EQUIPMENT STATIONS

(71) Applicants: United States Government as Represented by Department of Veterans Affairs, Washingyon, DC (US); The Texas A&M University System, Colleg Station, TX (US)

(72) Inventors: Lisa K. Cornelius, Round Rock, TX (US); Chetan Jinadatha, Belton, TX (US)

(21) Appl. No.: 18/020,771

(22) PCT Filed: Aug. 11, 2021
(86) PCT No.: PCT/US21/45536

§ 371 Date: Feb. 10, 2023

Related U.S. Application Data
(60) Provisional application No. 63/064,222, filed on Aug. 11, 2020.

(51) Int. Cl. A61B 90/00; A61G 10/00

(52) U.S. Cl. CPC A61B 90/05; A61G 10/005

(57) ABSTRACT

A mobile personal protective equipment (PPE) station can comprise a frame that is configured to be movable on a floor surface and a shield that is coupled to the frame. The shield can comprise a front wall that extends along a first horizontal axis. The front wall can define a pair of arm holes. At least a portion of the front wall can be transparent.

### CROSS-REFERENCE TO RELATED APPLICATIONS

This application claims priority to and the benefit of the filing date of U.S. Provisional Patent Application No. 63/064,222, filed Aug. 11, 2020, the entirety of which is hereby incorporated by reference herein.

### FIELD

This application relates generally to protective devices, assemblies, and systems for providing barriers between medical professionals and patients.

### BACKGROUND

The coronavirus (SARS-CoV-2) pandemic has infected over 20 million people worldwide with over 757,000 reported deaths. In the United States, over 3 million people have been infected with over 163,000 deaths reported and with cases rising daily. Prevention of healthcare-associated infections (HAIs) has been a priority in all hospitals around the world. Personal protective equipment (PPE) supplies are in acute shortage worldwide. Patients in a hospital can transmit to healthcare providers and other susceptible individuals via droplets and direct contact. Without appropriate PPE, healthcare workers could unknowingly transmit SARS-CoV-2 to uninfected patients.

Appropriate PPE to provide care for these patients includes isolation gowns, gloves, face shields, and N95 respirators. The gowns and face shields provide protection against droplets from patients when they cough and sneeze on a healthcare worker (for example, while the healthcare worker is providing care). Because of acute shortages of gowns and face shields, and in view of the ever-increasing cases and demand for PPE, the use of appropriate, safe and reusable alternatives to gowns and face shields is of paramount importance.

In further situations, radiologists (e.g., interventional radiologists), cardiologists, and certain hospital equipment operators often work long hours wearing a heavy, leaded jacket that provides very limited coverage (for blocking radiation) creating a major occupational hazard for these individuals. In particular, head, legs, toes, eyes, and other body parts are not covered by the apron or jacket. Further, because of the long-term effects from the weight of the lead aprons or jackets worn by these individuals, there is a need for an alternative to such lead aprons and jackets.

### SUMMARY

Disclosed herein, in one aspect, is an apparatus comprising a frame that is configured to be movable on a floor surface. A shield can be coupled to the frame. The shield can comprise a front wall that extends along a first horizontal axis. The front wall can define a pair of arm holes that are configured to receive arms of a healthcare worker positioned behind the front wall. At least a portion of the front wall can be transparent.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

These and other aspects of the invention will become more apparent in the detailed description in which reference is made to the appended drawings wherein:

FIG. 1 is front perspective view of a mobile personal protective equipment (PPE) station in accordance with embodiments disclosed herein.

FIG. 2 is a rear perspective view of the mobile PPE station of FIG. 1.

FIG. 3 is front perspective view of the mobile PPE station of FIG. 1 with the shield in a collapsed configuration.

FIG. 4 is a perspective view of a compartment of the mobile PPE station of FIG. 1.

FIG. 5 is a front perspective view of an exemplary mobile PPE station in accordance with embodiments disclosed herein.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a compartment” can include two or more such compartments unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” “generally,” “approximately,” or “substantially,” it is contemplated that values within up to 15%, up to 10%, up to 5%, or up to 1% (above or below) of the particularly stated value or characteristic can be included within the scope of those aspects.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

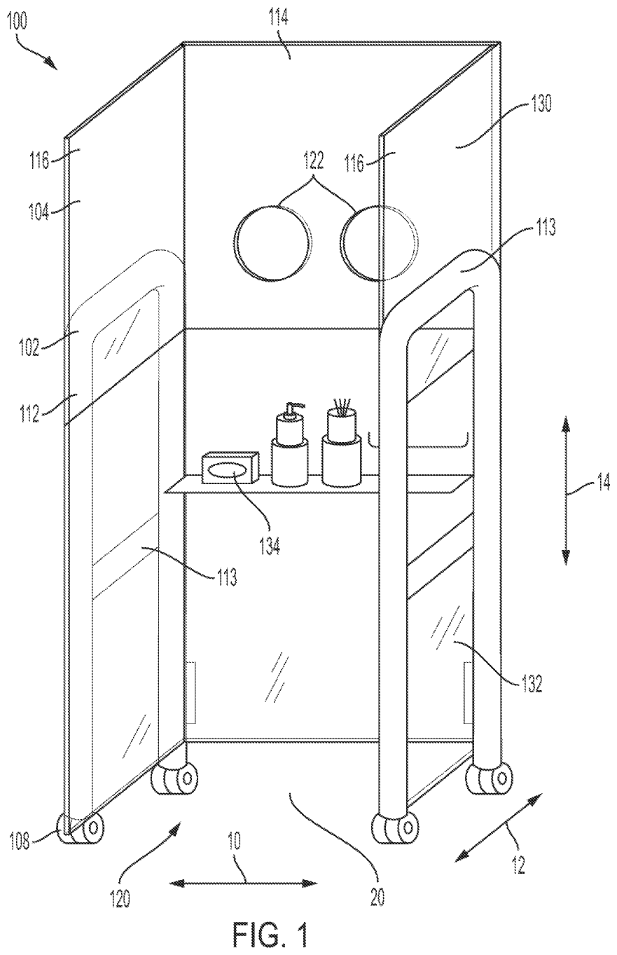

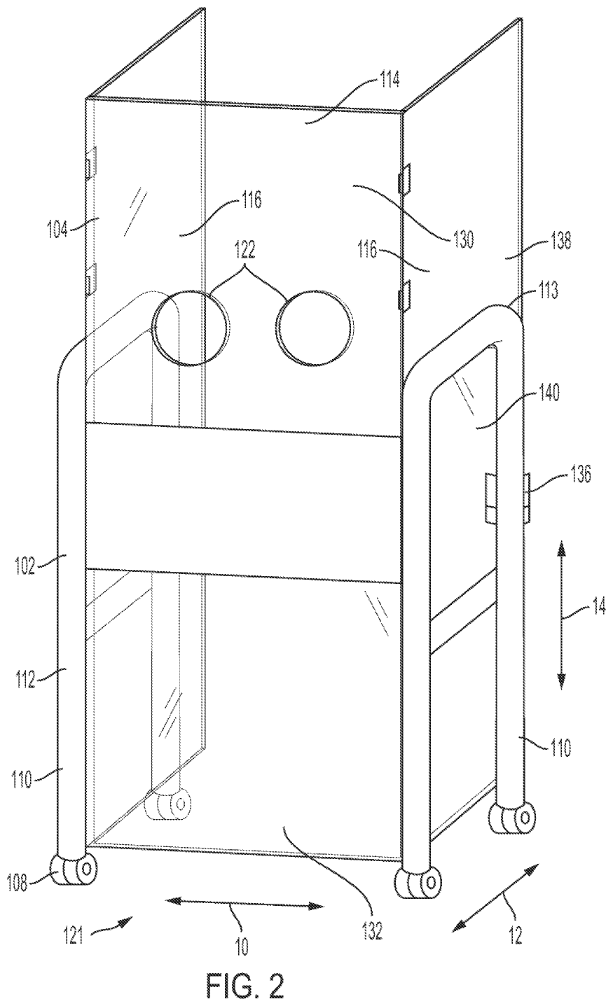

Disclosed herein, in various aspects and with reference to FIGS. 1-2, is a mobile PPE station 100 comprising a frame 102 and a shield 104 that is coupled to the frame 102. The frame 102 can optionally comprise two sides 110 that are spaced along a first horizontal axis 10. Each side 110 can have a plurality of (e.g., two) vertically extending legs 112 and transverse portions 113 (e.g., one transverse portion one each side) that extend between the legs 112. In some optional aspects, the frame 102 can be embodied as a walker, as a child walking aid, or a stroller. In this way, it is contemplated that existing products can be adapted for use as a portion of the mobile PPE station 100. However, in other aspects, the frame 102 can be specifically manufactured for inclusion in a mobile PPE station as disclosed herein.

The frame 102 can be configured to move along a floor surface 20. For example, in some aspects, the frame 102 can comprise wheels 108 (e.g., optionally, one at a bottom end of each leg 112). Optionally, one or more of the wheels 108 can be selectively lockable. In exemplary aspects, the wheels 108 can comprise caster wheels as are known in the art. In further aspects, the frame can comprise slidable skids. In some aspects, the slidable skids can be provided as an alternative to wheels 108. For example, skids can be positioned at portions of the frame that rest against the floor surface 20. In exemplary aspects, skids can comprise felt, hard polymer, or other material that facilitates sliding and inhibits scuffing. In still further aspects, it is contemplated that front legs or rear legs can be provided with wheels while the other of the front legs or rear legs can be provided with skids. In use, and as further disclosed herein, it is contemplated that a healthcare worker can selectively move the frame 102 along the floor surface 20 to position the frame 102 in a desired location (e.g., a location in proximity to a patient).

The shield 104 can comprise a front wall 114 that extends along the first horizontal axis 10. In some aspects, the front wall 114 can divide a clinician space 120 and a patient space 121 that are positioned on opposing sides of the front wall 114. Optionally, the front wall 114 can be planar. In further aspects, the front wall 114 can be curved. In these aspects, it is contemplated that the curvature can have a consistent radius of curvature or have a complex curvature, with multiple radii of curvature. The front wall 114 can optionally extend to the floor surface or near the floor surface 20 (e.g., within one inch, within two inches, within six inches, or within one foot of the floor surface).

The shield 104 can further comprise opposing side walls 116 that extend rearwardly from the front wall 114 along a second horizontal axis 12 that is perpendicular to the first horizontal axis 10. In these aspects, it is contemplated that inner surfaces of the front and side walls 114, 116 can define the clinician space 120, while outer surfaces of the front and side walls can define the boundary of the patient space. In some aspects, the front wall 114 can couple to each side wall 116 via one or more fasteners, such as, for example and without limitation, interlocking tongue and groove structures, adhesive material, clamps, hinges, and the like. Optionally, abutting or adjacent portions of the front wall 114 and side walls 116 can be sealed to minimize fluid communication between the clinician space 120 and the patient space. The side walls 116 can optionally be planar. In some optional aspects, the side walls 116 can be perpendicular or substantially perpendicular (e.g., within 15 degrees, within 10 degrees, within 5 degrees, or within 1 degree (either less than or greater than) of perpendicular) to the front wall 114. In further aspects, the front wall 114 and side walls 116 are unitarily formed as a single monolithic curved component. For example, optionally, the front wall 114 and side walls 116 can cooperatively define a rounded (e.g., optionally, hemi-cylindrical) surface.

Optionally, in further aspects, the side walls 116 can be omitted. For example, in some aspects, the shield 104 can be embodied entirely by the front wall 114.

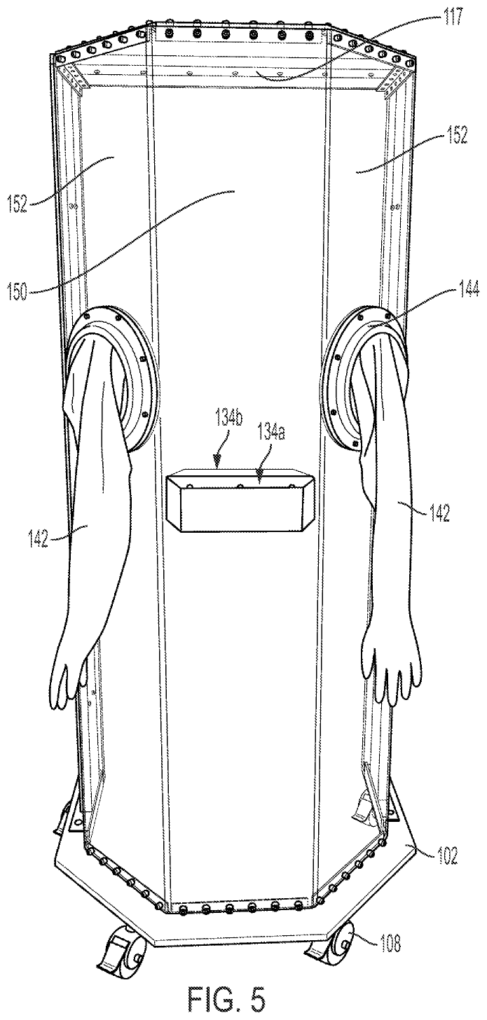

In embodiments where the side walls 116 are provided, the shield 104 can optionally comprise a rear wall (not shown) that is spaced rearwardly from the front wall portion along the second axis 12 and extends between the side wall portions 116 along the first axis 10. In further aspects, the shield 104 can comprise a top barrier 117 (FIG. 5) that extends rearwardly from the front wall along the second horizontal axis 12. Accordingly, in some aspects, the shield 104 can define the clinician space 120, which can correspond to an interior of the shield 104 between the front wall, side walls, and, optionally, the rear wall and top barrier. In aspects having a rear wall, at least one of the walls can define or comprise a door to provide access into the clinician space 120. Optionally, an entire wall (e.g., the rear wall) can be hingedly coupled to the frame so that the wall itself can serve as a door. In further aspects, a portion of wall (e.g., the rear wall) can serve as a door. The door can be selectively latched in the closed position and, optionally, latched in an open position. Optionally, the shield can define a port (optionally, a plurality of ports) for receiving air from a clean air source (e.g., a HEPA filter) and/or a conditioned air source. Accordingly, the mobile PPE station 100 can serve as a power air purifying respirator (PAPR). In some aspects, the port can be positioned at a top of the shield, and air can flow downwardly to an outlet space (e.g., between the ground 20 and a lower edge of the shield 104). Thus, air flow (optionally, laminar air flow) can direct air away from the head of the medical professional within the clinician space 120, inhibiting contaminated air from entering the interior 120 of the shield.

The front wall 114 of the shield 104 can define at least one pair of arm holes 122 that are configured to receive a medical professional's arms therethrough and allow mobility of the medical professional's arms when positioned within the arm holes 122. Optionally, the arm holes 122 can be circular. For example, the arm holes 122 can have a diameter from about six inches to about 12 inches. Optionally, the arm holes 122 can be spaced along the first horizontal axis 12 at approximately the shoulder width of an average adult (and have the same or substantially the same vertical height). For example, in some aspects, the arm holes 122 can be spaced center-to-center along the first horizontal axis 10 by from about 10 inches to about 20 inches, or from about 12 inches to about 18 inches. The arm holes 122 can be vertically spaced from an upper edge of the front wall 114 so that the upper edge of the front wall is over the head of the medical professional when her arms are extended through the holes 122. For example, the arm holes 122 can be at least 18 inches or at least 24 inches from the upper edge of the front wall along a vertical axis 14.

Optionally, with reference to FIG. 5, gloves 142 can couple to the shield 104 at the arm holes 122. For example, the gloves 142 can be configured to receive the medical professional's hands and at least a portion of (optionally, an entirety of) their arms. The gloves 142 can optionally be configured to elongate. For example, in some aspects, the gloves can comprise accordion folds. In some aspects, the gloves 142 can seal around the arm holes 122 so that air cannot pass therethrough. Optionally, the front wall 114 can comprise a flange coupling 144 (or other conventional coupling) that enables a sealing engagement between the gloves 142 and the shield 104 at the arm holes 122. For example, a proximal portion of the gloves 142 can be compressively engaged between the flange 144 and the shield 104 via screws or other fasteners. In further aspects, the gloves can be taped or clipped to the front wall or otherwise secured thereto. The gloves 142 can be decoupled for cleaning and disinfecting or replacement. In exemplary aspects, the gloves 142 can be isolation gloves as are known in the art. In these aspects, it is contemplated that the gloves 142 can comprise various materials, such as, for example and without limitation, rubber (e.g., neoprene, hypalon, butyl rubber, or the like).

In further optional aspects, sleeves without gloves can couple to the shield in a similar manner. Distal ends of the sleeves be configured to extend to and tighten down against the wrists of the medical professional so that the medical professional can wear separate (e.g., disposable) gloves. For example, an elastic element can extend around at least a portion of the circumference of the distal end of the sleeve to compress the distal end of the sleeve around the wrist, thereby preventing exposure of the skin of the clinician.

Optionally, in some aspects, it is contemplated that the mobile PPE station 100 can be dimensioned to accommodate more than one medical professional within the clinician space 120. In such aspects, it is contemplated that the front wall 114 can define two or more pairs of arm holes 122. Optionally, each hole 122 of each pair of arm holes can be provided with a respective glove 142 as disclosed herein. It is further contemplated that unused arm holes can be blocked/plugged (e.g., with removable a plug insert) to inhibit air and particulate transfer therethrough.

In some aspects, at least a portion of the shield 104 can extend to at least six, at least seven, or at least eight feet above the floor surface 20 along the vertical axis 14. At least a portion of the mobile PPE station 100 can be collapsible to permit quick reduction of an operative height of the mobile PPE station, for example, in order to enable the mobile PPE station 100 to pass through doorways or for compact storage. For example, in some aspects, front wall 114 can comprise an upper portion 130 and a lower portion 132. Optionally, the upper portion 130 can be movably coupled to the frame 102 so that the upper portion 130 is movable about and between a use position, in which the front wall extends a first distance above the floor surface along the vertical axis 14, and a collapsed position, in which the front wall extends a second distance above the floor surface along the vertical axis 14, wherein the first distance is greater than the second distance. For example, in some aspects, the upper portion 130 can be hingedly coupled to the frame 102 (and configured to fold downwardly). In further aspect, the upper portion 130 of the front wall 114 can be slidably coupled to the frame 102 (and configured to slide downwardly). Optionally, the upper portion 130 can slide along an outer side of the frame (e.g., between the frame and the patient). In further optional aspects, the upper portion 130 of the front wall 114 can be slidable by a user within the clinician area 120 (e.g., inside of the frame, with the upper portion being positioned between the frame and the medical professional). In various aspects, the frame 102 can comprise rails or tracks that facilitate sliding of the upper portion 130 of the front wall 114 (e.g., relative to the frame and the lower portion of the front wall). For example, the upper portion 130 can slide within tracks. Optionally, the upper portion 130 of the front wall 114 can be positioned forwardly of the lower portion 132. In further aspects, the upper portion 140 of the front wall 114 can be positioned rearwardly of the lower portion 132. Optionally, in a fully lowered/collapsed configuration, the upper portion 130 of the front wall 114 can be no greater than 5 feet, or no greater than 4 feet above the floor surface 20. In various aspects, in the fully lowered/collapsed configuration, the mobile PPE station 100 can have a height that is less than 75% or less than 65% or from about 50% to about 75% or about 55% to about 65% of the full height of the mobile PPE station when the mobile PPE station is not in the lowered/collapsed configuration.

More generally, it is contemplated that the operative height of the shield 104 can be selectively adjusted during use of the mobile PPE station 100. For example, in some aspects, in order to maintain an appropriate barrier between the patient and a caregiver positioned behind the front wall 114 (within the clinician space 120), at least a portion of the shield 104 (optionally, the entire shield) can be slidably coupled to the frame 102. In these aspects, it is contemplated that the operative height of the shield 104 can be selectively increased or decreased to account for the height of the patient and/or the caregiver (e.g., to position the arm holes 122 at a select height and/or to minimize the likelihood of transmission of fluid droplets between the patient and the caregiver). Optionally, as discussed above, it is contemplated that the front wall 114 can comprise an upper portion 130 and a lower portion 132. In further exemplary aspects, it is contemplated that the lower portion 132 can remain fixed to the frame 102, while at least a portion of the upper portion 130 can move relative to the frame. In these aspects, it is contemplated that the upper portion 130 can comprise a first panel that is fixedly secured to the frame 102 and a second panel that is configured to vertically slide relative to the frame and the first panel to increase the operative height of the front wall 114 as desired. It is contemplated that the position of the movable portion of the front wall 114 can be adjusted by releasing at least one releasable fastener and then reengaging the fastener when the desired position is achieved. Such releasable fasteners can include clamps, locking pins, hook and loop fasteners, screws, magnets, and the like. In further aspects, a lifting actuator can be configured to assist in adjusting the operative height of the shield 104. For example, a linear actuator, such as a pneumatic cylinder or electronic actuator can be operatively coupled to the shield 104 (or a movable portion of the shield) and configured for adjustment of the operative height of the shield. An input device (e.g., comprising a pair of up and down push buttons and/or buttons corresponding to preset heights) can be in communication with the lifting actuator and can be configured to enable an operator to select the operative height of the front wall 114. In yet further aspects, the upper portion 130 of the front wall 114 can fixedly couple to an upper portion of the frame, and the lower portion 132 can fixedly couple to a lower portion of the frame, and the upper and lower portions of the frame can be moved relative to each other to adjust the vertical position of the upper portion 130 of the front wall.

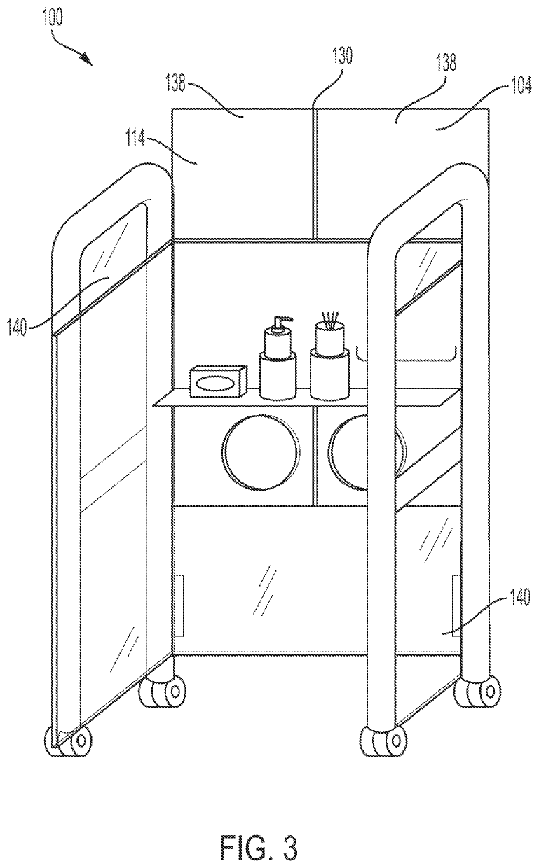

In further aspects, it is contemplated that the side walls 116 can comprise an upper portion 138 and lower portion 140. The upper portions 138 of the side walls 116 can be hingedly coupled to the upper portion 130 of the front wall 114 so that they can be pivoted from respective use positions (FIG. 1), in which the upper portions 138 of the side walls 116 extend from the front wall 114 along the second horizontal axis 12 (optionally, parallel to or generally parallel to (e.g., within 15 degrees, within 10 degrees, within 5 degrees, or within 1 degree of being parallel to) the second horizontal axis 12), to respective collapsed positions (FIG. 3), in which they are parallel to, or generally parallel to (e.g., within 15 degrees, within 10 degrees, within 5 degrees, or within 1 degree of being parallel to), the front wall. For example, the upper portions 138 of the side walls 116 can be folded outwardly until they are positioned in front of the front wall 114. In further aspects, the upper portions 138 of the side walls 116 can be folded inwardly so that they are positioned rearwardly of the front wall 114. With the upper portions 138 of the side walls 116 moved to their respective collapsed positions, the upper portion 130 of the front wall 114 can be slid downwardly (as shown in FIG. 3) or folded forwardly (e.g., 180 degrees or about 180 degrees about a horizontal pivotal axis) to a compact configuration to reduce the operative height of the shield 104. In further aspects, the upper portions 138 of the side walls 116 can remain in their use position (extending along the second axis 12) as the upper portion 130 of the front wall and upper portions 138 of the side walls 116 are slid downwardly, together (as a coupled assembly) or independently, to lower the height of the mobile PPE station 100. Optionally, the side walls 116 can be decoupled from the front wall 114 and/or frame 102 for repair or replacement.

In exemplary aspects, the upper portion 130 of the front wall 114 can be pivotable relative to the frame by a limited amount (e.g., less than 30 degrees from parallel, less than 15 degrees from parallel, or less than 10 degrees from parallel) to enable the medical professional to bend over the patient. It is contemplated that the limited amount by which the upper portion 130 is pivotable can be limited to an angle that prevents the mobile PPE station 100 from tipping over or otherwise being unstable.

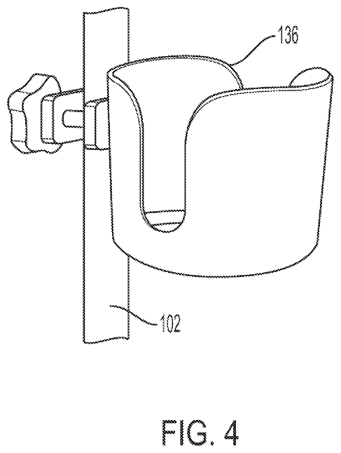

In some aspects, the mobile PPE station 100 can define at least one shelf 134 that is configured to hold cleaning supplies, wipes, gloves, goggles, medical supplies, and other patient care items. The shelf 134 can be positioned rearwardly of the front wall 114. In some aspects, when the upper portions 138 of the side walls 116 are pivoted to their respective collapsed positions, the upper portions 138 of the side walls 116 can be positioned forwardly of the shelf 134 so that the shelf does not interfere with lowering of the upper portions of the front and side walls of the shield. Optionally, the shelf 134 can be between about 6 and 8 inches deep along the second horizontal axis 12 and 2-3 feet in length along the first horizontal axis 10. For embodiments in which the shield 104 comprises curved surfaces, the depth of the shelf can be variable across its length. Optionally, in further aspects, the shelf 134 (or shelves) can be positioned to the side or sides (spaced from the center along the second horizontal axis 12) to avoid the need for the caretaker to lean over the shelf or shelves. Optionally, it is contemplated that the overall width of the mobile PPE station 100 can be increased to accommodate side shelves. In some aspects, the mobile PPE station 100 can comprise a door or flap that provides access to the shelf 134 (or shelves) within the clinician space 120 from the patient space 121. In some aspects, and with further reference to FIG. 5, the mobile PPE station 100 can define a first shelf 134a that is positioned within the patient space 121 on an outer side of the shield 104 and a second shelf 134b that is positioned within the clinician space 120 on an inner side of the shield. The first shelf 134a can be configured to hold supplies used on the patient (e.g., scalpels, syringes, etc.), whereas the second shelf 134b can be configured to hold supplies used by the medical professional (e.g., sanitizing supplies, wipes for sweat, etc.). In further aspects, and with reference to FIG. 4, the mobile PPE station can define or comprise at least one compartment 136. As shown in FIG. 5, in some aspects, the shelf or shelves 134 can comprise an outer wall that retains supplies on the shelf. Optionally, one or more compartments 136 can be coupled (e.g., removably or permanently coupled) to the frame 102. At least one compartment 136 can be sized to receive a disinfectant bottle or a container holding wipes. At least one compartment 136 can be sized to hold a box of gloves (e.g., 6 inches by 12 inches). The mobile PPE station 100 can further comprise a hook for hanging a mask or a compartment for holding the mask.

At least a portion of the shield 104 can comprise a transparent material. For example, the front wall can comprise polycarbonate or acrylic. In some optional aspects, portions of the shield (e.g., lower portions) can comprise opaque materials, for example, to reduce cost or to include materials with high durability. The shield 104 can comprise smooth surfaces in order to enable cleaning and disinfection. In further aspects, the shield 104 can comprise shatter-proof glass or impact-resistant glass, which can be particularly important for outdoor applications or for violent patients. Optionally, the frame 102 and/or the fasteners (that couple the frame together and the shield 104 to the frame) can comprise stainless steel (e.g., 304 or 316L stainless steel) for impact and weather resistance.

In further aspects, the shield 104 can comprise laminated lead glass (e.g., LPX-650 or LPX-700, manufactured by Lemer Pax, or glass of similar grade and protection). In these aspects, it is contemplated that the shield 104 can optionally comprise a top barrier and rear wall as further disclosed herein to define a closed compartment. In this way, a medical professional, such as a radiologist, can eliminate the need for wearing a lead apron or a lead jacket. Further, it is contemplated that the mobile PPE station 100 can provide protection against both radiation and droplets from patients.

Referring to FIG. 5, in various aspects, the front wall 114 of the shield 104 can have a central portion 150 and beveled side portions 152 that extend away from the central portion at obtuse angles. In some aspects, the arm holes 122 can be defined in respective beveled side portions 152 positioned on opposing sides of the central portion 150. In this way, the arm holes 122 can ergonomically receive the arms of the medical professional.

In some aspects, to enhance communication between a patient in the patient space 121 and a medical professional within the clinician space 120, a microphone can be positioned within the clinician space 120 to capture the voice of the medical professional. A speaker (optionally, coupled to the mobile PPE station 100) can be in communication with the microphone to broadcast the voice of the medical professional to the patient.

In some aspects, the edges of the shield 104 can be beveled or rounded. In some aspects, the mobile PPE station 100 can comprise one or more handles for moving.

In some aspects, the mobile PPE station 100 can comprise a temperature sensor (e.g., an infrared temperature sensor) that can enable the mobile PPE station to serve as a prescreening station.

In use, it is contemplated that the disclosed mobile PPE station 100 can address current shortages of PPE equipment by eliminating or reducing the need for gowns or eye protection. Additionally, it is contemplated that the mobile PPE station can continue to protect against droplets from patients even when a medical professional changes positions (e.g., turns around) while using the mobile PPE station, thereby providing a consistent level of protection as long as the medical professional stays behind the shield. In contrast, during the use of conventional PPE (i.e., gowns or eye protection), the medical professional can be significantly more exposed when he or she turns around or otherwise changes positions. For example, many gowns have open back designs that can create significant exposure.

Still further, the mobile PPE station 100 can improve efficiency by reducing PPE donning and doffing time. In yet further aspects, the mobile PPE station 100 can serve as a barrier between a clinician and a disruptive patient (E.g., a demented or confused patient) to prevent injury to the clinician from thrown objects, biting, or other attacks.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus comprising: a frame that is configured to be movable on a floor surface; and a shield that is coupled to the frame, the shield comprising: a front wall that extends along a first horizontal axis, wherein the front wall defines a pair of arm holes that are configured to receive arms of a healthcare worker positioned behind the front wall, and wherein at least a portion of the front wall is transparent.

Aspect 2: The apparatus of aspect 1, wherein the shield further comprises opposing side walls that extend from the front wall along a second horizontal axis that is perpendicular to the first horizontal axis.

Aspect 3: The apparatus of aspect 2, wherein the front wall is generally planar and the opposing side walls extend rearwardly from the front wall of the wall along the second horizontal axis.

Aspect 4: The apparatus of claim 3, wherein the opposing side walls are planar and parallel or generally parallel to the second horizontal axis.

Aspect 5: The apparatus of aspect 2 or aspect 3, wherein the front wall and side walls are unitarily formed as a single monolithic curved component.

Aspect 6: The apparatus of any one of the preceding aspects, wherein the front wall comprises an upper portion and a lower portion, wherein the upper portion is movable relative to the lower portion.

Aspect 7: The apparatus of aspect 6, wherein the upper portion of the front wall is configured to move to a fully collapsed position.

Aspect 8: The apparatus of aspect 7, wherein, when the upper portion of the front wall is in the fully collapsed position, the upper portion of the front wall is no greater than five feet from the floor surface.

Aspect 9: The apparatus of any one of aspects 6-8, wherein the shield further comprises opposing side walls, wherein at least a portion of each of the opposing front walls is pivotable about and between: a first position, in which the at least a portion of the respective side wall extends from the front wall along a second horizontal axis that is perpendicular to the first horizontal axis; and a second position, in which the at least a portion of the respective side wall is parallel to or generally parallel to the front wall.

Aspect 10: The apparatus of aspect 9, wherein the opposing side walls each comprise an upper portion and a lower portion, wherein the upper portion of each side wall is pivotable about and between the first and second positions.

Aspect 11: The apparatus of aspect 10, wherein the upper portion of the front wall and respective upper portions of the side walls are slidable along a vertical axis about and between a raised configuration and a compact configuration to reduce an operative height of the apparatus.

Aspect 12: The apparatus of aspect 10, wherein the upper portion of the front wall and respective upper portions of the side walls are pivotable about and between a raised configuration and a compact configuration to reduce an operative height of the apparatus.

Aspect 13: The apparatus of any one of aspects 2-13, wherein at least a portion of the front wall and at least a portion of the side wall are configured to slide vertically as a coupled assembly.

Aspect 14: The apparatus of any one of aspects 2-13, wherein the shield further comprises a rear wall that extends between the side walls along the first axis.

Aspect 15: The apparatus of any one of the preceding aspects, wherein the shield further comprises a top wall that extends rearwardly from the front wall along the second horizontal axis.

Aspect 16: The apparatus of any one of the preceding aspects, wherein the frame is movably supported on wheels.

Aspect 17: The apparatus of aspect 16, wherein at least one of the wheels is selectively lockable.

Aspect 18: The apparatus of any one of the preceding aspects, wherein the frame is movably supported on slidable skids.

Aspect 19: The apparatus of any one of the preceding aspects, wherein the frame comprises one of a walker, a child walking aid, or a stroller.

Aspect 20: The apparatus of any one of the preceding aspects, wherein the shield extends upwardly along a vertical axis at least eight feet from the floor surface.

Aspect 21: The apparatus of any one of the preceding aspects, wherein at least a portion of the shield is collapsible relative to the frame to reduce a height of the shield above the floor surface.

Aspect 22: The apparatus of aspect 21, wherein the front wall comprises an upper portion that extends above the frame, wherein the upper portion is hingedly coupled to the frame to reduce the height of the shield above the floor surface.

Aspect 23: The apparatus of aspect 21, wherein the front wall comprises an upper portion that extends above the frame, wherein the upper portion is slidably coupled to the frame so that the upper portion is movable along a vertical axis.

Aspect 24: The apparatus of any one of the preceding aspects, wherein the apparatus defines at least one compartment.

Aspect 25: The apparatus of any one of the preceding aspects, wherein the apparatus defines at least one shelf.

Aspect 26: The apparatus of any one of the preceding aspects, wherein the shield comprises polycarbonate or acrylic.

Aspect 27: The apparatus of any one of the preceding aspects, wherein the shield comprises laminated lead glass.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

1. An apparatus comprising:
a frame that is configured to be movable on a floor surface; and
a shield that is coupled to the frame, the shield comprising:
a front wall that extends along a first horizontal axis,
wherein the front wall defines a pair of arm holes that are configured to receive arms of a healthcare worker positioned behind the front wall, and
wherein at least a portion of the front wall is transparent.

2. The apparatus of claim 1, wherein the shield further comprises opposing side walls that extend from the front wall along a second horizontal axis that is perpendicular to the first horizontal axis.

3. The apparatus of claim 2, wherein the front wall is generally planar and the opposing side walls extend rearwardly from the front wall of the wall along the second horizontal axis.

4. (canceled)

5. The apparatus of claim 2, wherein the front wall and the opposing side walls are unitarily formed as a single monolithic curved component.

6. The apparatus of claim 1, wherein the front wall comprises an upper portion and a lower portion, wherein the upper portion is movable relative to the lower portion.

7. The apparatus of claim 6, wherein the upper portion of the front wall is configured to move to a fully collapsed position.

8. The apparatus of claim 7, wherein, when the upper portion of the front wall is in the fully collapsed position, the upper portion of the front wall has a height that is no greater than five feet.

9. The apparatus of claim 6, wherein the shield further comprises opposing side walls, wherein at least a portion of each of the opposing side walls is pivotable about and between:
a first position, in which the at least a portion of the respective side wall extends from the front wall along a second horizontal axis that is perpendicular to the first horizontal axis; and
a second position, in which the at least a portion of the respective side wall is parallel to or generally parallel to the front wall.

10. The apparatus of claim 9, wherein the opposing side walls each comprise an upper portion and a lower portion, wherein the upper portion of each side wall is movable about and between the first and second positions.

11. The apparatus of claim 10, wherein the upper portion of the front wall and respective upper portions of the side walls are slidable along a vertical axis about and between a raised configuration and a compact configuration to adjust an operative height of the apparatus.

12. The apparatus of claim 10, wherein the upper portion of the front wall and respective upper portions of the side walls are pivotable about and between a raised configuration and a compact configuration to adjust an operative height of the apparatus.

13. The apparatus of claim 2, wherein at least a portion of the front wall and at least a portion of each of the opposing side walls are configured to slide vertically as a coupled assembly.

14. The apparatus of claim 1, further comprising a plurality of wheels coupled to the frame, wherein the frame is movably supported on the plurality of wheels.

15. (canceled)

16. The apparatus of claim 1, wherein the frame is movably supported on slidable skids.

17. The apparatus of claim 1, wherein the frame comprises one of a walker, a child walking aid, or a stroller.

18. (canceled)

19. The apparatus of claim 1, wherein at least a portion of the shield is collapsible relative to the frame to reduce a height of the shield above the floor surface.

20. The apparatus of claim 19, wherein the front wall comprises an upper portion that extends above the frame, wherein the upper portion is hingedly coupled to the frame to reduce the height of the shield above the floor surface.

21. The apparatus of claim 19, wherein the front wall comprises an upper portion that extends above the frame, wherein the upper portion is slidably coupled to the frame so that the upper portion is movable along a vertical axis relative to the frame.

22. (canceled)

23. (canceled)

24. (canceled)

25. (canceled)

26. The apparatus of claim 2, wherein the shield further comprises a rear wall that extends between the side walls along the first axis.

27. (canceled)

28. The apparatus of claim 1, wherein the front wall comprises a central portion and opposing beveled side portions that extend away from the central portion at obtuse angles.
